(19) United States
(12) Patent Application Publication
(10) Pub. No.: US 2022/0161289 A1
(43) Pub. Date: May 26, 2022

(54) SYSTEMS, DEVICES, AND METHODS FOR DRINKING APPARATUS ORIFICE PROTECTION

(71) Applicant: Elkay Manufacturing Company, Downers Grove, IL (US)

(72) Inventors: Aaron Byers, Stockton, IL (US); Bryan Miller, Lemont, IL (US)

(21) Appl. No.: 17/532,825

(22) Filed: Nov. 22, 2021

Related U.S. Application Data
(60) Provisional application No. 63/117,402, filed on Nov. 23, 2020.

(51) Int. Cl. B05B 15/50; E03B 9/20; A61L 2/10

(52) U.S. Cl. CPC B05B 15/50; A61L 2202/11; A61L 2/10; E03B 9/20

(57) ABSTRACT

An orifice protection device for a water dispensing device is disclosed. The device includes a housing configured to surround an output orifice of the water cooler or fountain through which water is ejected, a door movably attached to the housing and configured to move from an open position in which the output orifice is exposed, and a closed position in which the output orifice is enclosed within the housing, an actuator configured to move the door between the open position and the closed position, a purification mechanism positioned within the housing and configured to sanitize the output orifice, and a sensor configured to enable touchless operation of the door.

### CROSS-REFERENCE TO RELATED APPLICATIONS

This application claims the benefit of U.S. Provisional Patent Application No. 63/117,402, filed Nov. 23, 2020, which is fully incorporated by reference in its entirety herein.

### TECHNICAL FIELD

The present disclosure generally relates to water coolers and/or fountains, bubblers, water bottler fillers, and other water or drink dispensing devices, and, more specifically, systems and methods for protecting the output orifices from contamination.

### BACKGROUND

Water coolers, also referred to herein as drinking fountains, typically include an exposed orifice through which water is ejected in an upward arc. A user of the cooler or fountain lowers his or her head toward the stream of water to take a drink. The close proximity of the user's mouth to the exposed orifice can lead to contamination of the orifice, the water stream, and/or the area surrounding the exposed orifice. Some water coolers or fountains may also or alternatively include a downward facing orifice which may be used to fill a water bottle. This orifice may be contacted by the lip or top of a user's bottle, which can lead to similar contamination of the orifice, the water stream, and/or the area surrounding the exposed orifice.

In certain water cooler devices, a water filtration system may operate to purify or clean the water before it is ejected out of the exposed orifice. However, this filtration system does not address contamination that is left behind on the exposed orifice or nearby the exposed orifice, which may be picked up by the water stream as it is ejected. A second user who operates the water cooler or fountain may then become contaminated by using the cooler or fountain, such as by the water stream picking up any contamination left behind by the previous user.

There is a need for systems and methods for protecting water coolers and fountains from contamination, and to protect users from contaminating each other via the cooler or fountain and the stream of water ejected by the exposed orifice.

### SUMMARY

The present disclosure summarizes aspects of some example embodiments and should not be used to limit the claims. Other implementations are contemplated in accordance with the techniques described herein, as will be apparent to one having ordinary skill in the art upon examination of the following drawings and detailed description, and these implementations are intended to be within the scope of this application.

Example embodiments are shown for an orifice protection device for a water dispensing device. In one example, the orifice protection device includes a housing configured to surround an output orifice of the water fountain through which water is ejected, a door movably attached to the housing and configured to move from an open position in which the output orifice is exposed, and a closed position in which the output orifice is enclosed within the housing, an actuator configured to move the door between the open position and the closed position, a purification mechanism positioned within the housing and configured to sanitize the output orifice, a sensor configured to enable touchless operation of the door, and an indicator light configured to indicate that the output orifice of the water fountain has been sanitized.

In some examples, the orifice protection device is configured to operate around a bubbler orifice of a water fountain. In further examples, the orifice protection device is configured to operate around a bottle filler orifice of a water fountain.

### BRIEF DESCRIPTION OF THE DRAWINGS

For a better understanding of the invention, reference may be made to embodiments shown in the following drawings. The components in the drawings are not necessarily to scale and related elements may be omitted, or in some instances proportions may have been exaggerated, so as to emphasize and clearly illustrate the novel features described herein. In addition, system components can be variously arranged, as known in the art. Further, in the drawings, like reference numerals designate corresponding parts throughout the several views.

FIG. 1 illustrates an example water fountain.

FIG. 2 illustrates an example water fountain including a first orifice protection device for the bubbler orifice and a second orifice protection device for the bottle filling mechanism.

FIG. 3A illustrates an example water fountain basin and an orifice protection device with a closed door, according to examples of the present disclosure.

FIG. 3B illustrates the example water fountain basin and orifice protection device of FIG. 3A, wherein the door of the orifice protection device is in an open position.

FIGS. 4A, 4B, and 4C illustrate three stages of operation of an example orifice protection device for use with a bubbler orifice.

FIGS. 5A, 5B, and 5C illustrate close up views of the example orifice protection device of FIGS. 3A, 3B, 4A, 4B, and 4C.

FIGS. 6A, 6B, and 6C illustrate three stages of operation of an example orifice protection device for use with a bottler filler orifice.

FIGS. 7A and 7B illustrate the orifice protection device of FIGS. 6A, 6B, and 6C with various coverings and components removed, so as to illustrate the internal components.

### DETAILED DESCRIPTION OF EXAMPLE EMBODIMENTS

While the invention may be embodied in various forms, there are shown in the drawings, and will hereinafter be described, some exemplary and non-limiting embodiments, with the understanding that the present disclosure is to be considered an exemplification of the invention and is not intended to limit the invention to the specific embodiments illustrated.

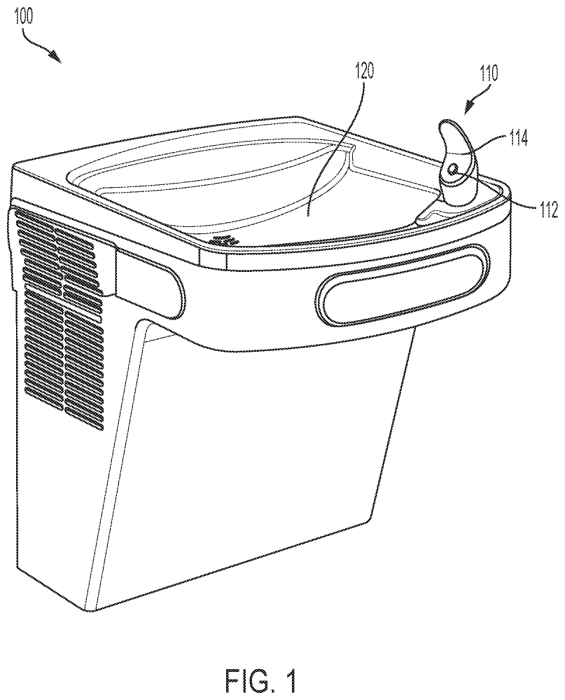

As noted above, a typical water cooler or fountain (which may also be referred to as a “drinking fountain” or “bubbler,” all of which are used interchangeably herein) can include multiple orifices through which water is ejected. A first orifice through which water is ejected for a user may be referred to herein as a bubbler orifice. For a bubbler orifice, water is typically ejected upward in an arc, so as to be available for consumption by a user. FIG. 1 illustrates a typical water fountain 100, including a bubbler orifice 110 and a basin 120. The bubbler orifice is illustrated having an exposed spout 112 and a guard 114. When used, the exposed spout 112 and/or guard 114 may become contaminated by various sources, including past users of the water fountain 100, passers-by, and by ambient or air-borne particles or droplets containing viruses or bacteria. Any virus, bacteria, or other such particles or impurities will be referred to herein collectively as “contamination.” When any contamination lands on the exposed spout 112, guard 114, or other surface near to where the water stream is ejected from the bubbler orifice 110, there is a possibility that the contamination is picked up by the water stream and may be transported from the water fountain 100 to a user of the water fountain 100. In this way, the water fountain 100 may act as a vector or source for spreading contamination within a community.

With the above issues in mind, a first example orifice protection device for a water fountain may be configured to protect the bubbler orifice and surrounding areas. In one example embodiment, the device includes an enclosure surrounding the exposed bubbler orifice 110 in combination with a disinfecting mechanism to prevent contamination from residing on the exposed bubbler orifice 110 and/or surfaces nearby or adjacent to the exposed bubbler orifice. In this example embodiment, the disinfecting mechanism is an ultraviolet (UV) radiation source within the bubbler orifice. The UV radiation source operates to clean the bubbler orifice and surrounding surfaces of the water fountain, thereby eliminating or rendering inactive the source of contamination. In some examples, the UV light source may be configured to emit a specific wavelength of light. For instance, the UV light source may be configured to emit UVA radiation (320-400 nm), UVB radiation (280-320 nm) and/or UVC radiation (200-280 nm). It should be appreciated that the light source may be configured to emit light having a multiple wavelengths, a single wavelength, and/or may be configured to emit light in one or more of ranges noted above and outside those ranges as well. In particular examples, UVC light (e.g., 200-280 nm) may be used due to the particular susceptibility of contaminants to this wavelength of light.

In certain embodiments, the disinfecting mechanism includes a UVC light source having a lower wavelength, such as a wavelength of 222 nm. In such embodiments, the UVC light source wavelength may be selected so that it is capable of disinfecting certain contaminants, while remaining relatively safe even when people are exposed to the light. This can allow for the bubbler or other water source to be purified without the need for a cover or housing to protect a user from the UV light source when in operation. For example, one source of radiation having a 222 nm wavelength is a krypton chloride excimer lamp. Furthermore, operation at this wavelength may enable a “low dose” of UV light to be used that effectively destroys contaminants. The low dose may be as low as or less than 2 mJ/cm². In some examples, the low dose may be as low as 1.3 mJ/cm². It should be appreciated that other amounts may be used as well.

In other embodiments, example orifice protection devices may also provide an actuator or movable door that opens and closes to respectively enable a user to drink from the bubbler orifice 110 when open, and to enable the disinfecting mechanism to operate without risk to the user when closed. Further, the example orifice protection device may provide for hands-free or partially hands free operation of the water fountain, which limits the likelihood of transmission of contamination from one user to another via the water fountain.

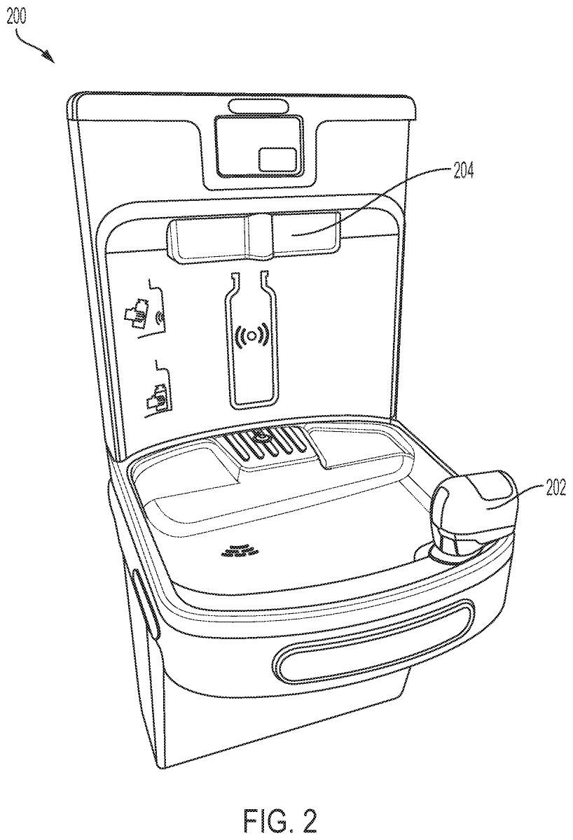

FIG. 2 illustrates an example water cooler or fountain 200 which includes two orifice protection devices. A first orifice protection device 202 is configured to protect the bubbler orifice and surrounding areas to reduce or eliminate contamination from user consumption. This example embodiment also includes a second orifice protection device 204, which is configured to protect the bottler filler orifice and surrounding areas. It should be appreciated that an example water fountain may include one or both of the illustrated orifice protection devices.

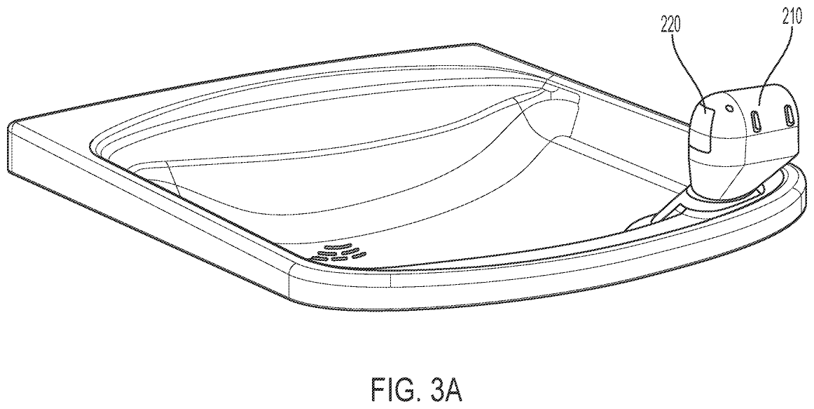

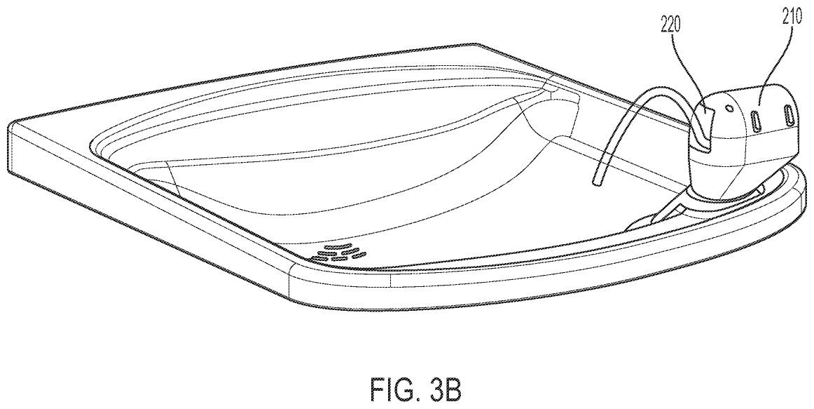

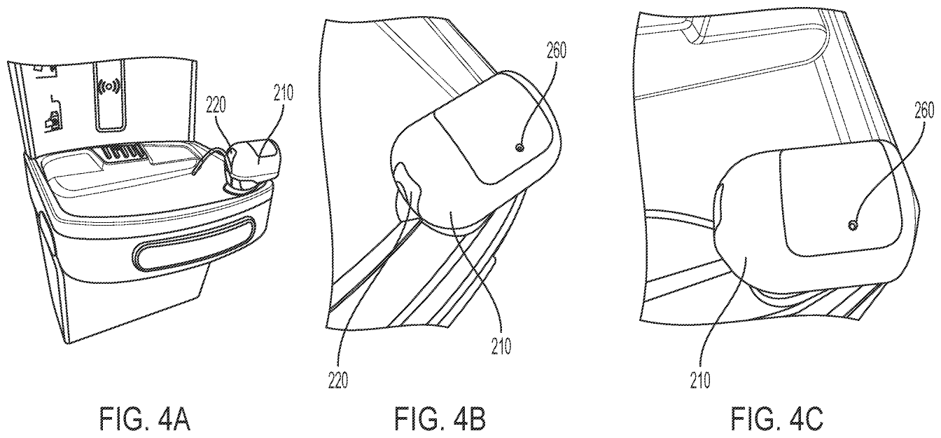

FIGS. 3A, 3B, 4A, 4B, 4C, 5A, 5B, and 5C illustrate example orifice protection device 202 as it may be installed on a water cooler or fountain such as water fountain 200. It should be noted, however, that the orifice protection device 202 may be installed on any suitable bubbler orifice, regardless of whether the bubbler orifice is connected to a larger water fountain. The example orifice protection device 202 may include a housing 210 surrounding the exposed bubbler orifice of the water fountain, a door 220 attached to the housing 210, an actuator 230 configured to move the door 220 between a closed position (e.g., FIG. 3A) and an open position (e.g., FIG. 3B), a purification mechanism 240 positioned inside the housing 210 and aimed at the exposed bubbler orifice 112 of the water fountain, and a sensor 250 configured to detect the presence of a user. The orifice protection device 202 may also include a display mechanism 260 configured to alert a user when the bubbler is ready to be used.

The housing 210 may be configured to entirely or partially replace an existing guard of a bubbler orifice. In some examples, the housing 210 may be manufactured in two parts, which fit together to fully or partially enclose the exposed bubbler orifice 112, as well as one or more of the other various components or parts of the device (e.g., the actuator 230, purification mechanism 24, sensor 250, and/or display mechanism 260). In some examples, the housing 210 may be made from any suitable material, such as plastic, metal, an alloy, or any other suitable material. The material selected may be such that it has antimicrobial or anti-contamination properties that make it easier for the material to be cleaned and remain contamination free.

One or more sections or parts of the housing 210 may be made from a material or manufactured such that this section enables the sensor 250 to work properly. For instance, the housing 250 may include a small opening through which the sensor 250 is aimed such that a motion of a user may be sensed. Alternatively or additionally, the material of the housing may be selected or configured such that it is transparent or semi-transparent in a particular spectrum used by the sensor 250. This may enable the sensor to operate effectively while remaining enclosed by the housing 210.

The housing 210 may also be made from a material that is opaque to UV light, and/or whatever the particular light that is used by the purification mechanism. This may prevent the UV light from the purification mechanism 240 from leaking out and potentially causing harm.

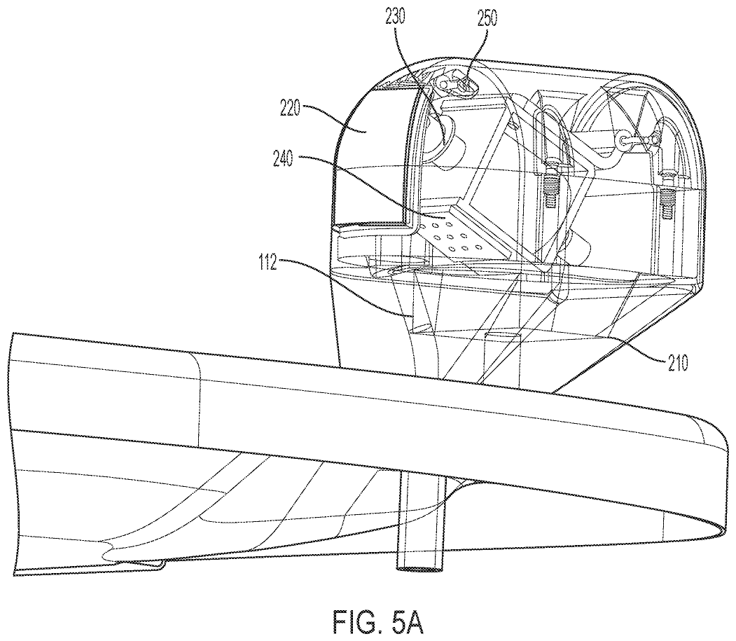

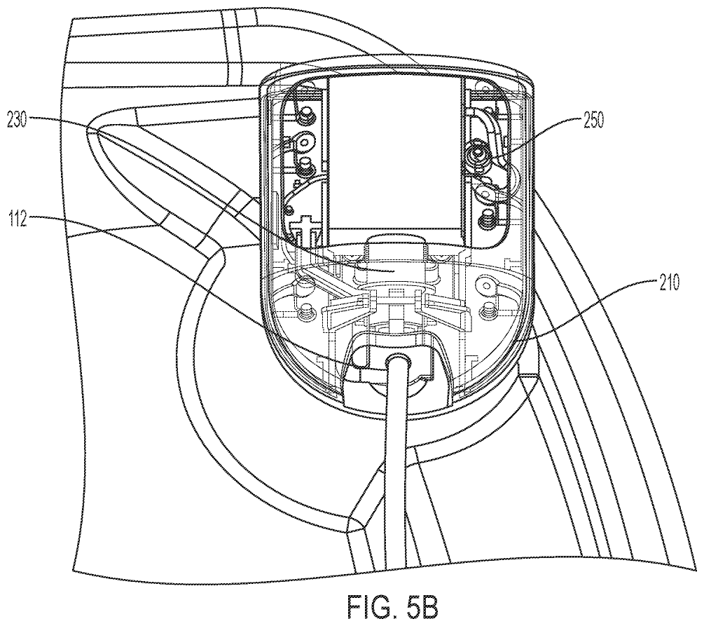

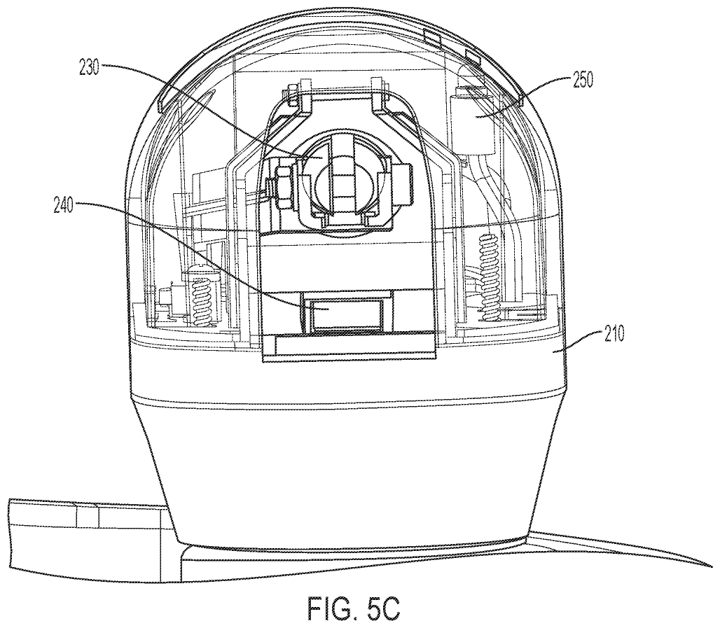

The door 220 may be configured to open and close automatically. This automatic operation may be caused by signals received from the sensor 250. In some examples, the door 250 may be generally rectangular in shape, and/or may include a curve that follows the contours of the housing 210 as shown in FIG. 5A. It should be noted, however, the that shape, size, and other aspects of the door 220 may be any suitable shape, size, etc., and that the specific design shown in the Figures if for example only.

In some examples, the door 220 may be attached to the housing 210 via a hinge located at a top of the door 220, oriented such that the door 220 swings inward into the interior of the housing 210. However, it should be understood that the door may be hinged on a different side or edge of the door 220, the door 220 may swing outward from the housing 210 rather than inward, or some other door mechanism may be used. For instance, the door may instead comprise two or more pieces which each have a hinge or connection mechanism. Further, the door 220 may be configured to slide open (similar to a garage door), rotate open (similar to an iris of a lens), or open using any other suitable mechanism or method of operation.

In the embodiment shown in the figures, the door 220 is configured to open inwardly to the housing 210, such that the water stream ejected by the bubbler orifice 112 is blocked when the door 220 is closed, and the bubbler orifice 112 is not blocked when the door 220 is open.

The door 220 may be attached to an actuator 230, which may be configured to move the door 220 between an open position and a closed position. In alternative embodiments, the door 220 may be configured to move between an open position and a closed position by any variety of methods well known in the art.

The actuator 230 in the illustrated embodiment is a solenoid that is attached to the door 220. The actuator 230 rotates in order to open and close the door 220. In the embodiment shown in FIG. 5A, the actuator 230 is mounted in the housing 210 at an angle with respect to the basin of the water fountain.

In some examples, the actuator 230 may also be configured to cause the water stream to be ejected by the bubbler orifice 112. This can include the actuator causing a mechanical operation such as causing a button to be pressed, or causing an electrical signal to be passed to the appropriate mechanism. Alternatively, the water fountain may include a separate actuator or control mechanism configured to activate the water stream from the bubbler orifice 112.

The purification mechanism 240 may be configured to disinfect or inactivate any contamination that may be present. To operate effectively, the purification mechanism may be attached to the housing 210 and positioned internally. In the example shown in the Figures, the purification mechanism 240 comprises an ultraviolet LED (e.g., UVC LED) that is aimed at the bubbler orifice 112 and various nearby or surrounding surfaces. The light emitted by the LED may act to disinfect the external surface of the bubbler orifice 112, and any water sitting on nearby surfaces within the housing 210.

The purification mechanism 240 may be configured to activate automatically for a predetermined amount of time responsive to the door 220 transitioning from an open state to a closed state. In an alternative embodiment, the purification mechanism 240 may be configured to be manually activated for any period of time as determined by the user. It should be appreciated that in alternative embodiments, both the activation and the duration of the activation of the purification mechanism 240 may be determined by the user.

The sensor 250 may be a hands free sensor configured to determine when a user has approached the water fountain and intends to take a drink. In some examples, the sensor 250 may be a light sensor aimed at a position where the user typically stands while using the water fountain. When a change in status occurs, the sensor 250 may trigger one or more functions such as an opening of the door 220, dispensing of water from the bubbler orifice 112, and more. When the user is done and leaves the area (and/or the sensor 250 detects a change in status), the door 220 may be configured to close and the purification mechanism 240 may be configured to activate to clean the area.

The display mechanism 260 may be an LED or indicator light that changes status based on one or more factors. In some examples, the indicator light may be lit when the device has performed a sanitization and the water fountain is ready to be used. After each use, the indicator light may be unlit for a period of time during which purification or sanitization is performed. This may be, for example, five seconds. After the period of time has elapsed (and the purification system has been activated for the period of time), the indicator light may again be lit to indicate that the water fountain is ready to use.

In some examples, the water fountain may be operable by user rather than being entirely hands-free. For instance, a mechanical mechanism (such as a bar on the front of the water fountain) may still be pressed or activated by a user to cause the door 220 to be opened and water to be ejected from the bubbler orifice.

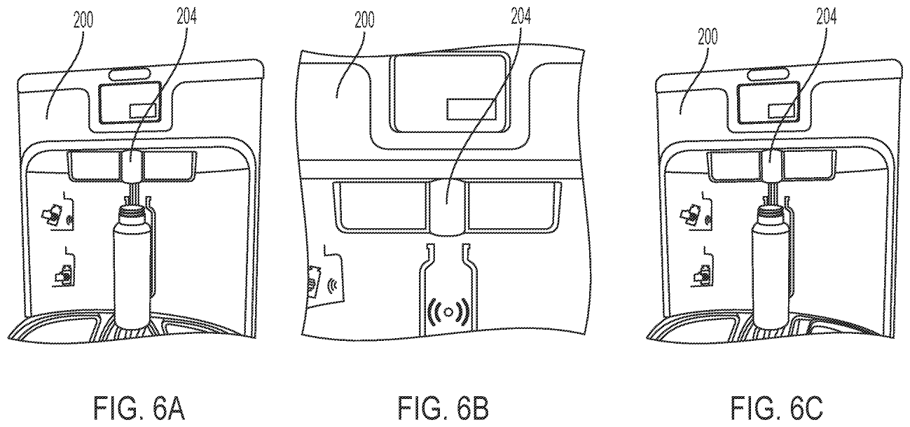

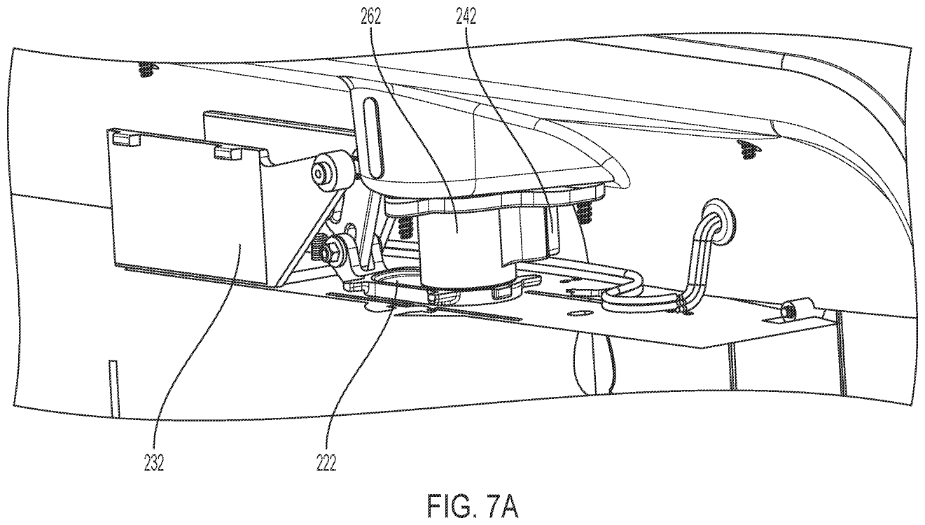

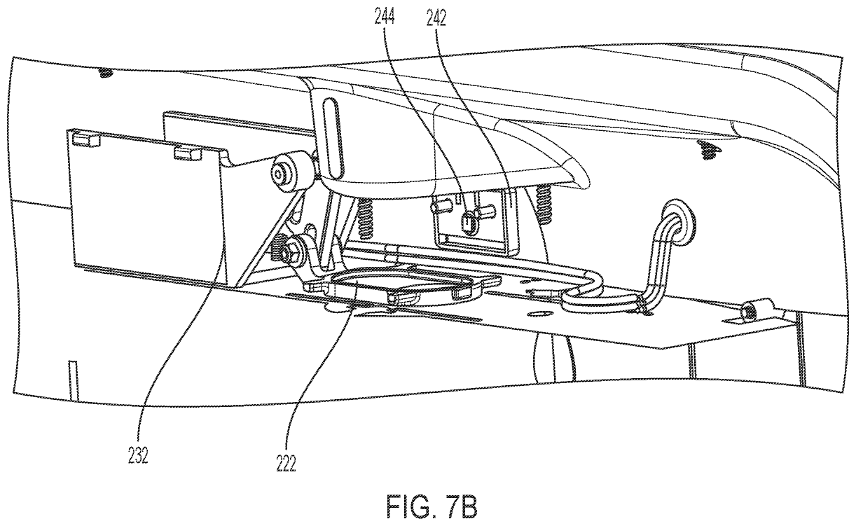

FIGS. 6A, 6B, and 6C, 7A, and 7B illustrate a second embodiment the orifice protection device described herein, shown as orifice protection device 204 for the bottle filler orifice. Orifice protection device 204 may be similar or identical to orifice protection device 202 in one or more respects. As such, any description herein with respect to orifice protection device 202 may also apply to orifice protection device 204 and vice versa.

The bottle filler orifice of the water fountain 200 may include a downward facing spout, configured to eject water into a water bottle positioned below the spout as shown in FIGS. 6A and 6C.

The orifice protection device 204 may include a housing, door, actuator, purification mechanism, sensor, and/or display mechanism configured to operate in a manner similar to those described above with respect to FIGS. 3A, 3B, 4A, 4B, 4C, 5A, 5B, and 5C. In some examples, the housing of the orifice protection device 204 may extend downward from an exterior surface of the water fountain 200 as shown in FIGS. 6A, 6B, and 6C. This may prevent the user from being exposed to and UVC light from the purification mechanism.

FIGS. 7A and 7B illustrate the orifice protection device 204 of FIGS. 6A, 6B, and 6C, but with various components removed to enable the internal components to be viewed.

FIGS. 7A and 7B illustrate that the orifice protection device 204 includes an actuator 232 configured to engage a door 222, The door 222 is configured to slide laterally to open and close the opening through which water is ejected downward into a bottle. FIG. 7 also illustrates a structural member 262 having a cylindrical inner opening through which water passes. The orifice protection device 204 also includes a purification mechanism 242 positioned at a back of the device 204, having an LED 244 positioned to shine light into the inner cylindrical opening of the structural member 262.

As noted above, some embodiments of the present disclosure may not include a housing or other covering mechanism for the purification mechanism (e.g., UV light source). For instance, some embodiments may include a UVC light source configured to emit light having a particular wavelength that is safe for humans, but is destructive or harmful to certain contaminants. This wavelength may be 222 nm, which corresponds to the low end of the UVC spectrum range. In this case, an example orifice protection device for a water dispensing device such as a bubbler, water fountain, or bottle filler may include a housing coupleable to the water dispensing device and a purification mechanism coupled to the housing, the purification mechanism configured to sanitize an output orifice of the water dispensing device. In some examples, the purification mechanism comprises an ultraviolet light source oriented to expose the output orifice of the water dispensing device to emitted ultraviolet light. Additionally, the UV light source may be configured to emit ultraviolet C (UVC) light, and in some examples specifically UV light having a wavelength of 222 nm. Further, the ultraviolet light source may be configured to emit light at an intensity of less than 2 mJ/cm².

In this application, the use of the disjunctive is intended to include the conjunctive. The use of definite or indefinite articles is not intended to indicate cardinality. In particular, a reference to “the” object or “a” and “an” object is intended to denote also one of a possible plurality of such objects. Further, the conjunction “or” may be used to convey features that are simultaneously present instead of mutually exclusive alternatives. In other words, the conjunction “or” should be understood to include “and/or”. The terms “includes,” “including,” and “include” are inclusive and have the same scope as “comprises,” “comprising,” and “comprise” respectively.

The above-described embodiments, and particularly any “preferred” embodiments, are possible examples of implementations and merely set forth for a clear understanding of the principles of the invention. Many variations and modifications may be made to the above-described embodiment(s) without substantially departing from the spirit and principles of the techniques described herein. All modifications are intended to be included herein within the scope of this disclosure and protected by the following claims.

What is claimed is:

1. An orifice protection device for a water dispensing device comprising:
a housing configured to surround an output orifice of the water dispensing device, the output orifice configured to eject a stream of water;
a door movably attached to the housing and positioned to block the stream of water, wherein the door is configured to move from an open position in which the output orifice is exposed, and a closed position in which the output orifice is enclosed within the housing;
an actuator coupled to the door and configured to move the door between the open position and the closed position; and
a purification mechanism positioned within the housing and configured to sanitize the output orifice.

2. The orifice protection device of claim 1, wherein the housing is configured to surround a spout of a bubbler orifice of the water dispensing device.

3. The orifice protection device of claim 1, wherein the housing is configured to surround a spout of a bottler filler orifice of the water dispensing device.

4. The orifice protection device of claim 1, wherein the housing is attachable to the water dispensing device, and wherein the orifice protection device is configured to receive power from the water dispensing device.

5. The orifice protection device of claim 1, wherein the door is positioned to block the stream of water ejected by the output orifice when in the closed position.

6. The orifice protection device of claim 1, wherein the door is coupled to the housing on a first side, and wherein the door is configured to move from the closed position to the open position and from the open position to the closed position by pivoting along the first side.

7. The orifice protection device of claim 1, wherein the door is configured to translate from the open position to the closed position, and translate from the closed position to the open position.

8. The orifice protection device of claim 1, wherein the actuator is positioned inside the housing.

9. The orifice protection device of claim 1, wherein the purification mechanism comprises an ultraviolet light source.

10. The orifice protection device of claim 9, wherein the ultraviolet light source is configured to emit ultraviolet C (UVC) light.

11. The orifice protection device of claim 10, wherein the ultraviolet light source is configured to emit light having a wavelength of 222 nm.

12. The orifice protection device of claim 10, wherein the ultraviolet light source is configured to emit light at an intensity of less than 2 mJ/cm².

13. The orifice protection device of claim 1, further comprising an indicator light coupled to the housing, wherein the indicator light is configured to indicate that the output orifice of the water fountain has been sanitized.

14. An orifice cleaning device for a water dispensing device comprising:
a housing coupleable to the water dispensing device; and
a purification mechanism coupled to the housing, the purification mechanism configured to sanitize an output orifice of the water dispensing device.

15. The orifice cleaning device of claim 14, wherein the purification mechanism comprises an ultraviolet light source oriented to expose the output orifice of the water dispensing device to emitted ultraviolet light.

16. The orifice protection device of claim 15, wherein the ultraviolet light source is configured to emit ultraviolet C (UVC) light.

17. The orifice protection device of claim 16, wherein the ultraviolet light source is configured to emit light having a wavelength of 222 nm.

18. The orifice protection device of claim 16, wherein the ultraviolet light source is configured to emit light at an intensity of less than 2 mJ/cm².
